(19) United States
(12) Patent Application Publication
(10) Pub. No.: US 2010/0292622 A1
(43) Pub. Date: Nov. 18, 2010

(54) Hip brace and hip brace kit

(76) Inventors: Andrea Carol Weissleder, Sarasota, FL (US); William Ross Kennedy, Sarasota, FL (US)

Correspondence Address: Andrea C. Weissleder, PT;William Ross Kennedy, MD, 4909 Featherbed Lane, Sarasota, FL, 34242, US

(21) Appl. No.: 12/454,375

(22) Filed: May 18, 2009

(51) Int. Cl. A61F 5/00

(52) U.S. Cl. 602/23

(57) ABSTRACT

Hip brace for motion control and support of the hip joint, and/or to the surrounding sacro-iliac joint and/or lumbo-sacral joints concomitantly, built of configured elasticized neoprene with tensile strength, and with closures built of velcro whose combined forces impart dynamic action and protection from hip dislocation and subluxation; range of motion control, joint protection and support, pain management, proprioceptive cueing, alignment support, resistive and assistive exercise options for people with disorders of the hip joint and as well for those with concomitant sacro-iliac and lumbo-sacral instability. Medical conditions addressed include but not limited to hip dislocation/subluxation, arthritis, sprains, strains, bursitis, tendonitis, joint hypermobility, ligamentous tears and sacro-iliac and/or lumbo-sacral pain and instability, as well as for athletes in need of hip joint and/or the sacro-iliac, lumbo-sacral joint protection and support on and off the field and for everyday ADL and exercise.

### BACKGROUND

When the hip joint is painful, vulnerable, weak, or subject to dislocation, the bracing devices most commonly used to this date are constructed of rigid or semi-rigid materials: metal uprights and leather over metal bands or plastic materials; often an exterior mechanical joint that allows only moderate free motion from a neutral position. These braces do not allow for any rotational hip range of motion. They are uncomfortable and difficult to manage; often subjecting a patient to blisters and sores from skin pressure from the firm brace materials pressing onto the skin. Many patients end up not using them. Sitting poses comfort issues with the rigid components, where as this device holds the US, SA and hips a comfortably supported in sitting position.

Other designs have used elasticized materials and velcro closures or like materials 1,2, albeit not in this proposed design and pattern (FIG. 1,2,3,4) for the body of the brace and application using velcro and optional ancillary neoprene or like material and velcro straps 1,2,10, FIG. 7,9 for positioning and support in these recommended configurations (FIG. 1-22). There is a need for a comfortable, effective brace of soft materials; whereas the brace is constructed of the soft, yet firm neoprene, or like elasticized fabric with tensile strength, cut into a specific pattern (FIG. 1,2,3,4,5) wrapped around the lower torso at the waist 3 with the neoprene or like elasticized fabric with tensile strength bands coming together in a specific direction 14 and positioned and overlapping to fit snuggly (FIG. 5) and then secured with velcro hook and loop straps 2,10,15, or like material pulling dynamically 14 in specific directions as per needed application. The thigh band 4 of same construction is wrapped around the thigh of the effected leg 11 in a specific configuration for each individual's fit and for the individual's deficit and purpose, be it general pain management, ambulation support, exercise (FIG. 15,16,17) containment of hot, cold or compression packs 19 (FIG. 9), to address specific muscles or muscle groups, to adjust to the strength or lack thereof, the neoprene velcro ended straps 2,18 (or like materials) can be applied 17 strategically to the body of the brace. The straps are adhered in a particular configuration as per rationale. (FIG. 1-22) The properties of elasticity and tensile strength of the neoprene or like fabric of the brace body itself (FIG. 1,2,3) the firm, but adjustable velcro closures (FIG. 8,9) and stretch in the direction of pull 14 of the additional neoprene velcro ended straps (FIG. 7,8,14) adjusted to the individual's contour and adhered to the brace body 14,15,16 determines the security of the hip joint, as well as the stability of the sacro-iliac and lumbo-sacral joints as the hip joint function and position is influenced by the security of the other surrounding joints—sacro-iliac and lumbo-sacral joints. (FIG. 18,19,20,21) 32,30,33. These joints are also secured with this brace using additional neoprene strap or straps of any needed length or width as cut to size is easy, with velcro closures with an alligator configuration 18 placed around the ends of a neoprene strap and then strategically onto the body of the brace 5.

### PREVIOUS ART

### U.S. Patents

| | | | |
|---|---|---|---|
| 4,901,710 | February 1990 | Meyer | 128/78 |
| 4,905,678 | March 1990 | Cumins and | 128/78 |
| | | Randolph | |
| 4,926,845 | May 1990 | Harris | 128/78 |
| 5,267,928 | December 1993 | Barile, Sawa | 482/124 |
| 5,419,755 | May 1995 | Fukumoto et al | 602/23 |
| 5,840,050 | November 1998 | Lerman | 602/19 |
| Des. 403071 | December 1998 | Biedermann, | D24/190 |
| | | Buchlholz | |
| 6,210,353 B1 | April 2001 | Barnes | 602/19 |
| US2002/0032397 A1 | March 2002 | Coligado | 602/5 |
| 6,540,703 B1 | April 2003 | Lerman | 602/5 |
| 2006/0015049 | January 2006 | Suarz, | 602/23 |
| | | Rosendahl | |
| 2006/0074365 A1 | June 2006 | Brown | 602/24 |
| US20040116260 | June 2004 | Drennan | 602/19 |
| US2007/0027419 A1 | February 2007 | Drennan | 602/19 |

### Rigid and Semi-Rigid Types of Braces:

| | | | |
|---|---|---|---|
| 4,901,710 | February 1990 | Meyer | 128/78 |
| 4,905,678 | March 1990 | Cumins and | 128/78 |
| | | Randolph | |
| 5,419,755 | May 1995 | Fukumoto et al | 602/23 |
| Des. 403071 | December 1998 | Biedermann, | D24/190 |
| | | Buchlholz | |
| 6,540,703 B1 | April 2003 | Lerman | 602/5 |
| 2006/0015049 | January 2006 | Suarz, | 602/23 |
| | | Rosendahl | |

The main differentiating aspects among the devices listed above include: use of leather covered metal or leather waist, hip and thigh cuffs, sockets or girdles of plastic or metal and utilizing metal uprights and metal motion calibrators designed to limit and set allowable hip motion. None, however, allowed for any rotational motion. None allowed for normal gait motion. Historically, these braces are often not tolerated. Older people get blisters and the result can be non-compliance.

### Semi Rigid:

Lerman U.S. Pat. No. 5,840,050 has similar components as that described in this patent application, but the components are “semi-rigid”, including some malleable metal stays connecting the thigh band to the waist band. The bands are not contoured to the body of the individual, although the metal stays can be shaped.

Barnes U.S. Pat. No. 6,210,353 B1 shows a different design using a short reach to buttocks support, metal components and an inflatable bladder.

Coligado US2002/0032397 uses a pelvic girdle of metal with a screw system for size adjustment.

### Soft Materials:

In examination of previous art, there are obvious changes in more recently available materials. The use of elasticized fabric like neoprene and closure systems of velcro have expanded the options for hip care.

Barile makes good use of the softer materials with a focus or rehabilitation, not for the immediate post surgical patient, i.e.; dislocators. Nevertheless, there is an elaborate, albeit cumbersome use of neoprene straps and velcro attachments. However, the attachments are sewn on various places onto a short pant. Velcro must go to these locations only or there is no way to secure a neoprene strap. The pant itself is too short and closures too flimsy, which compromises the mechanics. With a longer lever arm available only if the pant is longer, it is mechanically more advantageous to facilitate motion or motion control. The pants and thigh openings are cinched with a draw string, thus eliminating any waist band security and support for the pelvis; ie: sacro-iliac and L/S spine. Harris U.S. Pat. No. 4,926,845 is a pelvic exercise system that really addresses only the sacrum during exercise.

### The Closest in Search are:

Brown US2006/0074365 A1

This device is soft, has a dazzling variety of elasticized straps and uses velcro closures onto specific areas on the brace. The waist and thigh pieces are completely separate which eliminates the option to control the S/I and L/S, whose security is key to good hip joint rehabilitation. Strap tension must be high to hold the pelvis firmly, performed best with longer straps; i.e., lever arm. If the components are soft, the pelvis will tilt, unless better secured. The position and doubled fabric design of the closures would make it difficult for a patient to adjust if providing the needed tension.

Drennan US 2007/0027419 and its predecessor US20040116260 show a soft device with an elaborate system of loops and cables. The cables can be drawn through the multiple longitudinal rows of loops embedded into a neoprene waist band, thigh cuff (s) and a multiple number of elastic cables. A lateral strip joins the waist and thigh components together. There is a double layer of soft fabric to go against the skin and the neoprene or like material on the outer side. The cables operate a bit like a marionette, exerting forces to align the thigh to the hip in a desired position. The 2007 entry adds shoulder straps to help control the migration of the thigh piece toward the waist. Cables used with or without a covering of neoprene or other such fabrics are likely to be poorly tolerated by the wearer. A broader, flatter band distributes the forces better. The cables can also course over bony prominences. The number of possible position options is lengthy and cumbersome. He proposes an alternate fastening system using zippers. Neither one of the Dennan devices concomitantly support the S/I and L/S.

Generally, none of these devices address the sacro-iliac and L/S spine support needed in comprehensive hip care. Only the rigid devices claim a capability for controlling dislocation/subluxation.

For the rigid braces that claim anti-dislocation capability:

There is only the positional benefit from the rigid device. There is no discussion of tolerance, ambulation capability and absence of rotational capability.

None propose an exercise capability from the rigid device itself.

### For the Soft Braces:

None reveal positioning for control of dislocation/subluxation

None have used double sided UBL (unbroken loop) accepting velcro hooks on all parts of the brace. This capability enhances the function of the brace, including making it reversible for use on the other hip.

None describe a means to prevent strap slippage.

None grasp the concept for constant exercise availability while wearing a comfortable and effective support. None have described how the device fits if the straps are too long or too short.

None, however, allowed for any rotational motion. None allowed for normal gait motion.

Historically, these braces are often not tolerated. Older people get blisters and the result can be non-compliance.

### Semi Rigid:

Lerman U.S. Pat. No. 5,840,050 has similar components as that described in this patent application, but the components are “semi-rigid”, including some malleable metal stays connecting the thigh band to the waist band. The bands are not contoured to the body of the individual, although the metal stays can be shaped.
Barnes U.S. Pat. No. 6,210,353 B1 shows a different design using a short reach to buttocks support, metal components and an inflatable bladder.
Coligado US2002/0032397 uses a pelvic girdle of metal with a screw system for size adjustment.

### FIGURES

For purposes of this document, the drawings address the right hip, right sacro-iliac, right lumbo-sacral areas. Obviously, left sided configuration is the same with left sided orientation. The following brace configurations are examples of straight forward anatomical placement. Many other possible configurations, including proprioceptive neuromuscular facilitation patterns can be made to suit individuals, albeit using the same brace components.

FIG. 1: neutral

FIG. 2: side view

FIG. 3: posterior view

FIG. 4: pattern

FIG. 5: donning

FIG. 6: bilateral

FIG. 7: neoprene straps-kit components 38

FIG. 8: alligator velcro kit component 38;

FIG. 9: double sided patches, circular placement indicators applied to brace pattern 38

FIG. 10: moderately adducted thigh

FIG. 11: anterior view-anti-dislocation

FIG. 12: posterior view-anti-dislocation

FIG. 13: posterior view internally rotated

FIG. 14: anterior view internally rotated

FIG. 15: side view-assist or resist abduction

FIG. 16: flexion assist or extensor resist

FIG. 17: extensor assist or flexion assist

FIG. 18: sacro-iliac

FIG. 19: S/I and L/S (lumbo-sacral) strap position

FIG. 20: combined sacro-iliac and lumbo-sacral strap position reach to anterior

FIG. 21: exemplary use of several components used simultaneously

FIG. 22: rotation of the entire brace

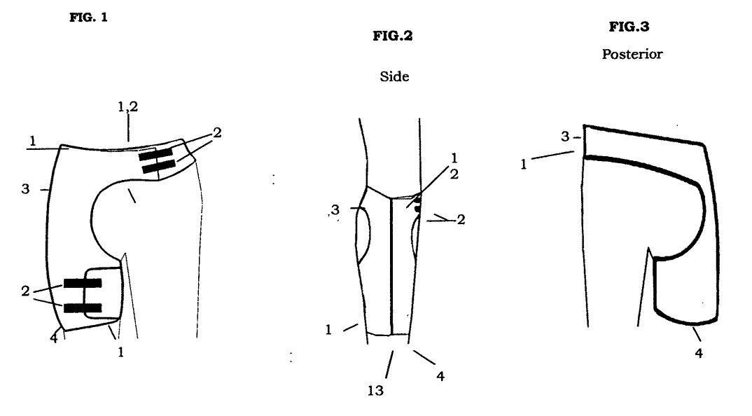

### DESCRIPTION OF FIGS. 1-22

### FIG. 1

### Neutral

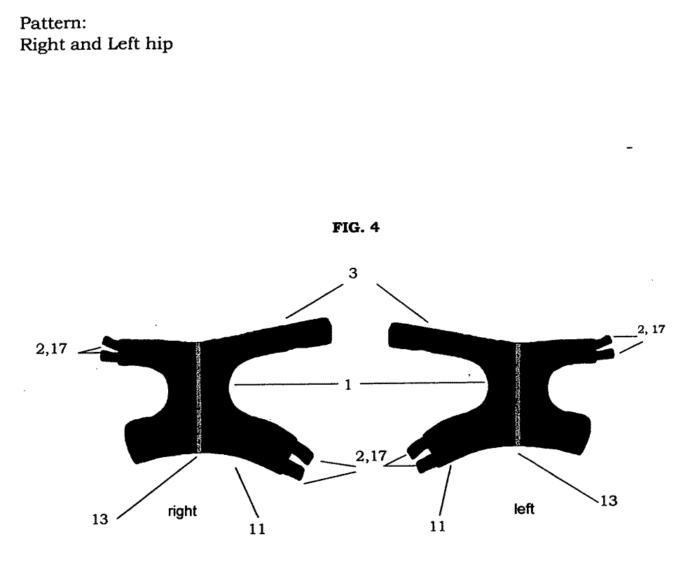

Drawing one depicts the embodiment of the hip brace in neutral position, configured for one (or 2-sided (FIG. 4)) neoprene body of the brace constructed in 2 parts and seamed together or cut from one piece and wraps around the lower torso and thigh. (FIG. 1,5) The body of the brace is constructed of elasticized neoprene or any generation of elasticized fabric with tensile strength, which is secured with velcro closures or like material or closure system 2. The placement and tension of four velcro straps and neoprene waist and thigh bands 12,13 2 as well as the compression onto the entire area covered by the brace, dictate the position of the thigh in reference to the pelvis and therefore the hip joint, sacroiliac, and lumbo-sacral spine. 12,3,2,4,11,13

### FIG. 2

### Side View

The side view of the right hip showing the position of the lateral seam aligned in midline. This is the beginning position when first donning the brace. 1,3,13,11,12,2

### FIG. 3

### Posterior View

The posterior portion of the brace stretches over the individual's sacrum and reaches distally to contain the sacro-iliac joint and courses down to the thigh. The problematic hip is covered by the neoprene held under tension by the dynamic tensile nature of the elasticized neoprene or like fabric and the velcro closures or other closure system. 1,2,3,4

### FIG. 4

### Pattern

The reversible pattern of the neoprene body 1, (FIG. 1) of the brace is shown with the removable velcro straps 2,17, on the ends of the waist and thigh bands 3,4,13,11,12 with both surfaces being receptive to the hook and loop of the velcro 2,10,17 or like material or other closure system. Thus, once flipped and velcro removed and reversed, it fits the other hip. Or, it can be made singularly as right or left and lined with soft fabric, although unnecessary, as the UBL surface is very soft. For close customization for individual people, the neoprene or other elasticized fabric can be cut proportionally to follow the pattern (FIG. 4). The velcro strap length can also be cut. 3,4

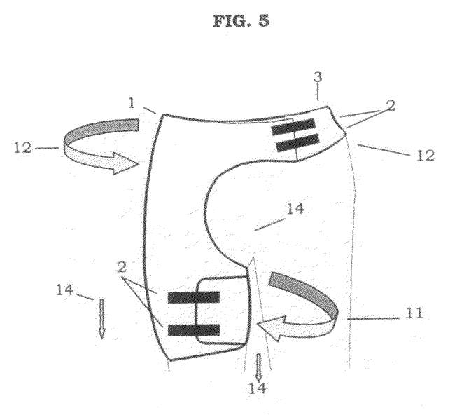

### FIG. 5

### Donning

Donning the hip brace body as in FIG. 1:

Utilizing the properties of elasticity, tensile strength, a neoprene or other like fabric and velcro hook and loop construction or like closure system, the brace is put on 1,2 by wrapping and drawing the top waist 3 band 12 around the waist while simultaneously stretching it firmly in the direction shown while keeping the midline seam 13 traced upon the side. The distal end of the waist band with the velcro hook end 2 is then pressed onto the body of the brace at the waist or just below. The thigh band 4 is then drawn and wrapped firmly around the thigh 11 fixing the end onto the front of the thigh portion 5. Subsequently, the thigh neoprene 4 piece is the pulled down distally 14 where the thigh circumference narrows and retightened there.

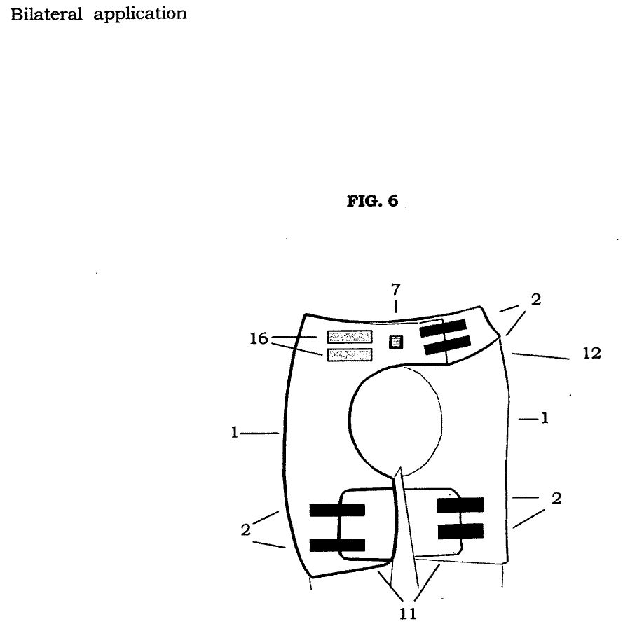

### FIG. 6

### Bilateral

Two braces can be applied simultaneously to each side if both sides have problems, even if those problems are different. (FIG. 6) or a customized bilateral single brace using the same configuration 16,1,2,11,12,7 using a common waist band.

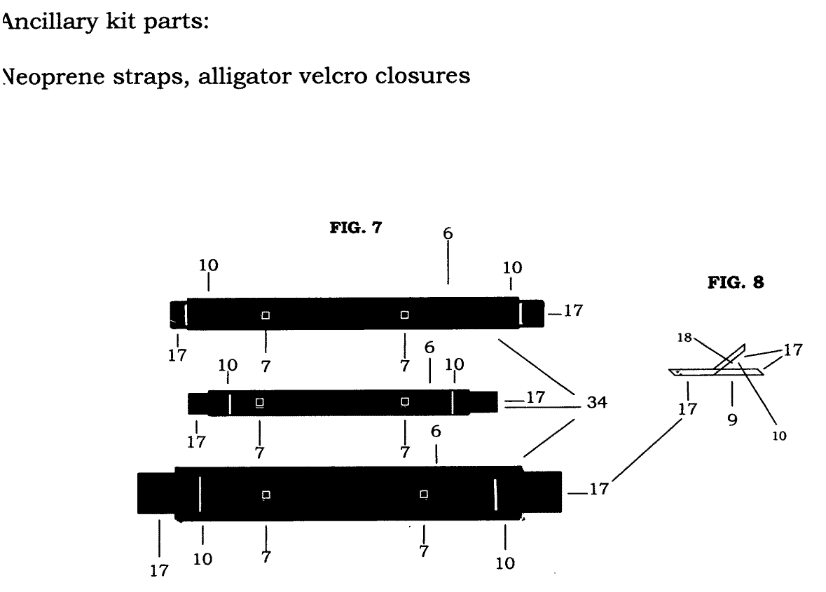

### FIG. 7

### Kit Component 38

Depicts samples of the neoprene ancillary straps, 6 or like material with properties of elasticity and tensile strength with velcro hook and loop ended straps (FIG. 7,8) or other closure system. The hip brace body hereto described is receptive to the adherence of velcro 10,17 anywhere on the surface. These ancillary straps serve to fine tune positioning and supportive adjustments by being stretched and attached to various configurations that stabilize, assist, resist, lift, and secure desired position (FIG. 11-21). The length of the straps is easily shortened by cutting the neoprene or velcro fabrics to the desired length. These ancillary straps can be used singly or in combination. All velcro is totally removable 10 allowing for precise adjustment and exercise capability. The amount of adjustment depends on placement and stretch 14 as well as coverage. They can be varying widths and lengths 34, stretched minimally to maximally.

### FIG. 8

### Alligator Type Velcro Closures; Kit Component 38

To make the brace totally adjustable, the velcro that attaches to the body of the brace is the double-ended velcro alligator mouth 17,9,18. It wraps around ends of the waist band and the thigh band, as well as the ancillary straps 18, 10. The velcro alligator type closures can be moved and re-attached for positional adjustment or switching it around for the other hip. 10

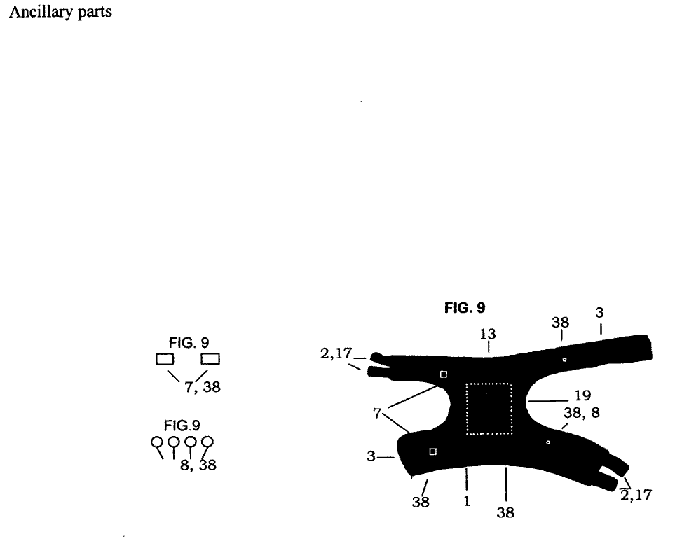

### FIG. 9

### Kit Components 38

Double-sided patches of velcro 7, 17 are used anywhere on neoprene to hold straps or parts of the bands from slipping. When a patch is placed, the sticky hook sides adhere to the bottom of one component to the top of the other. No undesired movement of the straps occurs and therefore holds in place for the desired purpose and adjustment for the time wearing the brace 35.

Circular velcro patches 8 are placed strategically onto the body of the brace to avoid confusion and remind the individual where the optimum placement 10,11,12,13,15,16,17,19,20 of the ancillary neoprene with velcro closure straps 2,5, or any velcro placement can be marked with a dot. A fabric pouch 19 made of soft, breathable, material and adhered onto the neoprene brace with velcro. Inside the pouch can be hot, cold, or compression packs or pads, magnets, electrodes, compression pad and other optional therapies. A pouch can be worn outside as well to contain small belongings.

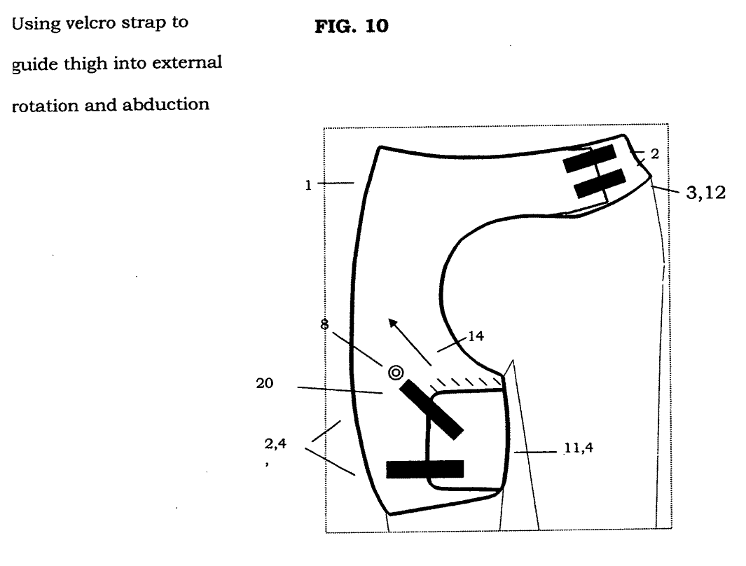

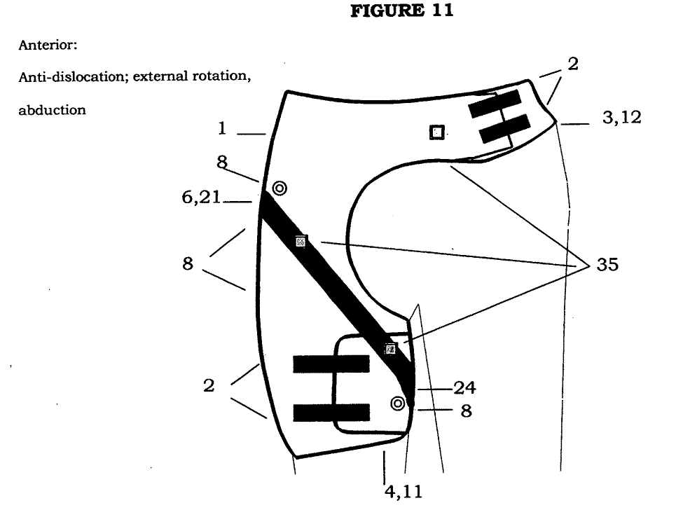

### FIG. 10

### Moderately Adducted Thigh

For the hip that moderately internally rotates or adducts in flexion, the superior thigh velcro 2,5, is applied at a 45″ angle, gently drawing the thigh into abduction and external rotation. The resulting gait will be more toward neutral.

### FIG. 11

### Anterior View-Anti-Dislocation

For the dislocating or subluxing hip, especially post surgically; bio-mechanically, the hip must be positioned in external rotation and abduction. Starting in neutral (FIG. 1) An ancillary neoprene strap 6 with velcro closures 2, or like materials is placed on the distal medial surface and drawn up and across the thigh to attach 5 on the waist band 4 on the opposite back side.

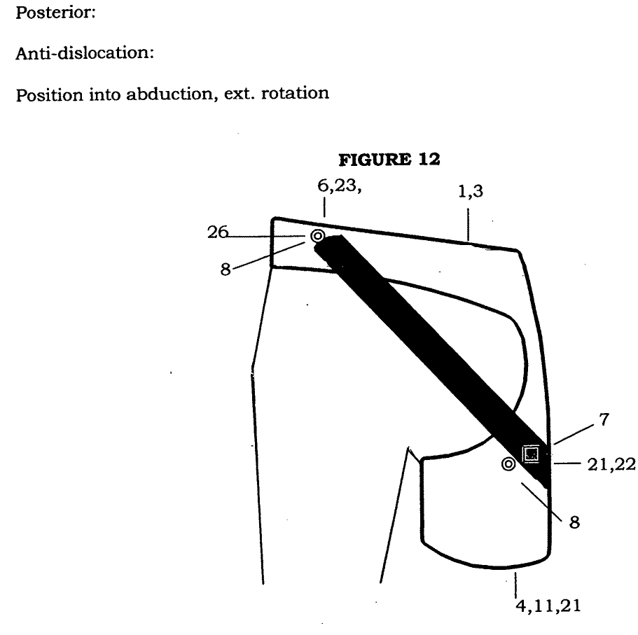

### FIG. 12

### Posterior View-Anti-Dislocation

Viewing FIG. 11 from the posterior, the attachment of the ancillary strap with velcro ends is indicated with the dot marker. The strap is stretched and pulled upward 13, 22 and attached on the waist band left of center. The provocative position for dislocation is prevented by this configuration. 1,3,12,8,6,7,11

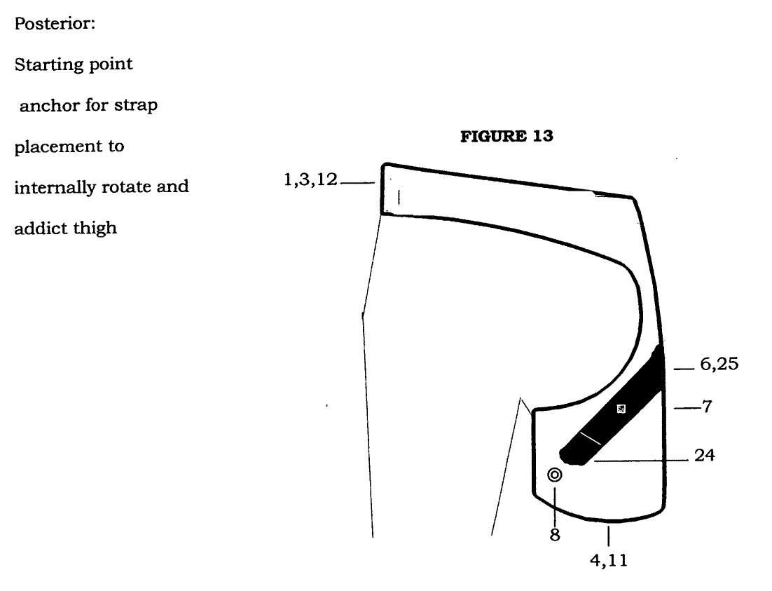

### FIG. 13

### Posterior View Internally Rotated

For the internally rotated hip, an ancillary velcro ended strap, or like materials, is first placed on the posterior medial lower thigh wrap 24; stretched pulling anteriorly and toward the opposite anterior side. 6,8,3,12,26,7,25,4,11

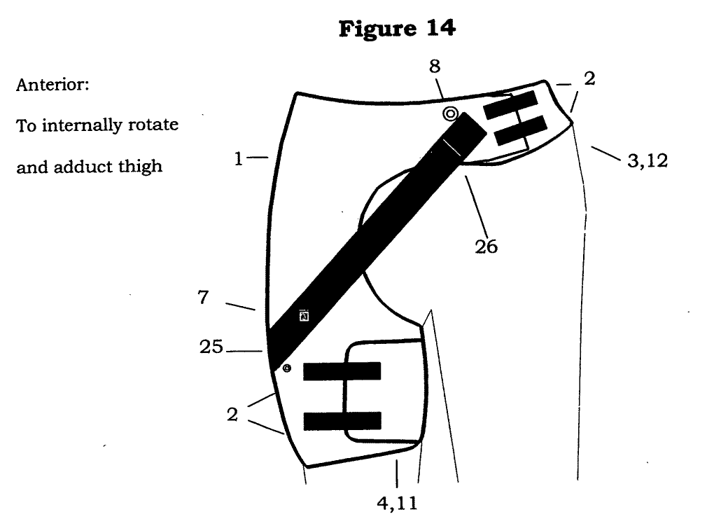

### FIG. 14

### Anterior View Externally Rotated to be Pulled into Neutral or Slight Internal Rotation

Continued stretch of the neoprene strap FIG. 13 is pulled anteriorly and upward to attach upon the waist band to the left of center where marked by the dot marker 8,4,11,24,7,6,25 guiding the thigh into more internal rotation. Under the strap is a double-sided velcro patch 7 to prevent movement of the strap.

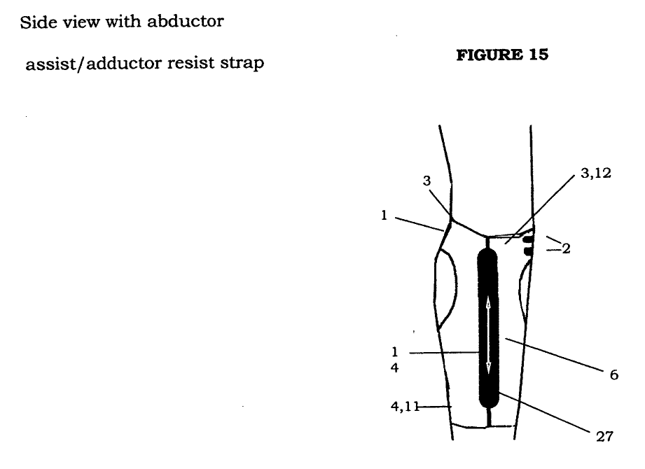

### FIG. 15

### Side View-Assist or Resist Abduction

A neoprene strap with velcro-ended strap (FIG. 7) is placed firmly on the lateral iliac crest stretched to the distal end of the lateral side. The tension produced by the pull of the fore mentioned strap assists the leg into abduction for those with weakness of hip abductors or, as an exercise resisting adduction, thereby strengthening the adductors. 27,14,2,4,11,12

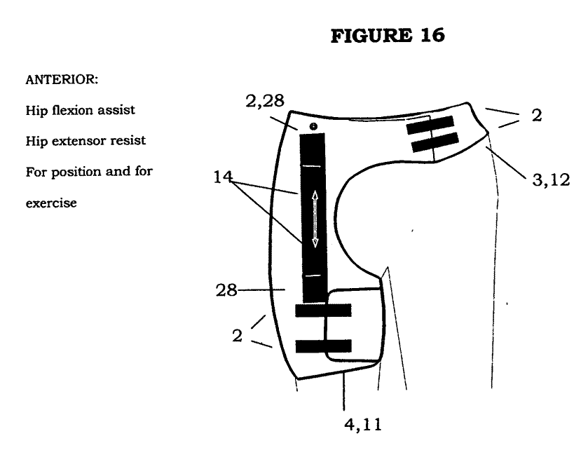

### FIG. 16

### Flexion Assist or Extensor Resist

An ancillary neoprene strap ((FIG. 7) or like material with velcro 2 or like closure material is anchored on the anterior iliac crest 3 along the anterior of the waist band 12 and stretched 14 to just above the velcro on the anterior thigh 4 and adhered there 11. The direction of pull acts to pull 14 the thigh capitally 28.

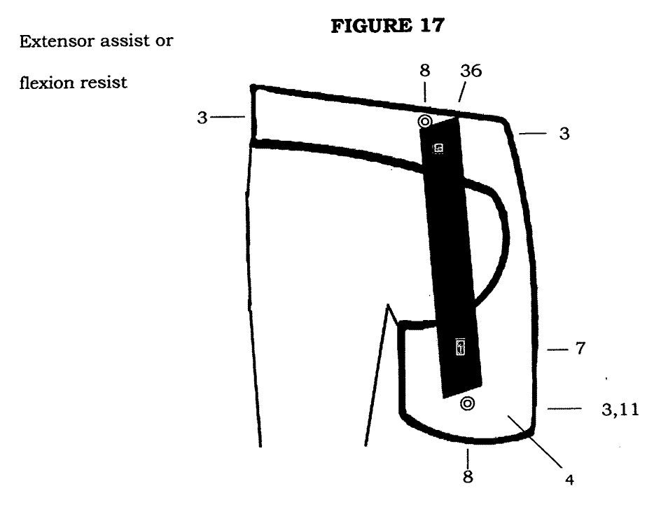

### FIG. 17

### Extensor Assist or Flexion Assist

An ancillary neoprene (FIG. 7) or like strap is anchored with velcro 2 onto the posterior waist 3 band 36 and stretched 14 caudally and adhered to the lower portion of the posterior thigh 4 band 11,7.

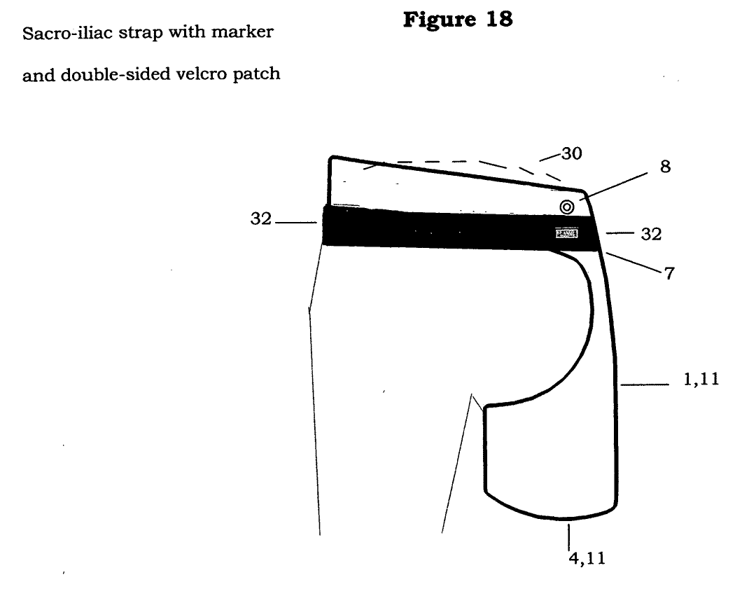

### FIG. 18

### Sacro-Iliac Strap

An ancillary neoprene strap with velcro ends 6 or other like materials is stretched 14 across the S/I joints, reaching around to adhere onto the medial or medial frontal area on the waist 3 band.

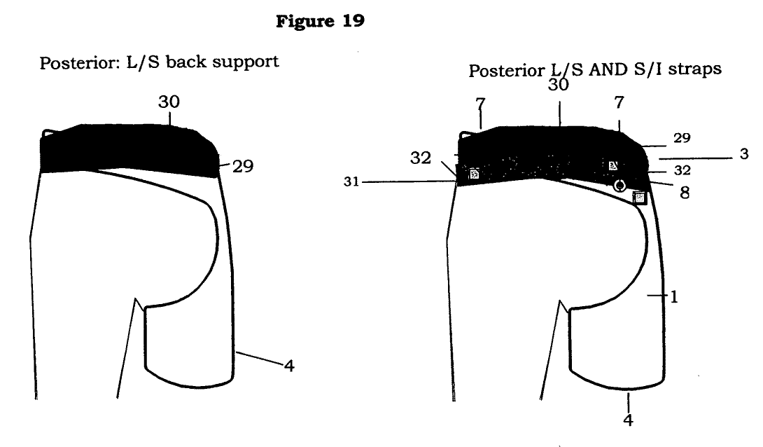

### FIG. 19

### Lumbo-Sacral Strap Position, US Plus S/I Together

Each of two neoprene ancillary straps 6 with velcro closures 5 or like materials applied one at a time; one anchored on the upper, L/S and the other on the lower waist band S/I 3 and drawn around the back to attach on the other side. The exerting pull and direction of tension is to support the sacro-iliac joints and lumbo-sacral junction. (FIG. 18,19) 32,6,30,8,7,1,4,29,14

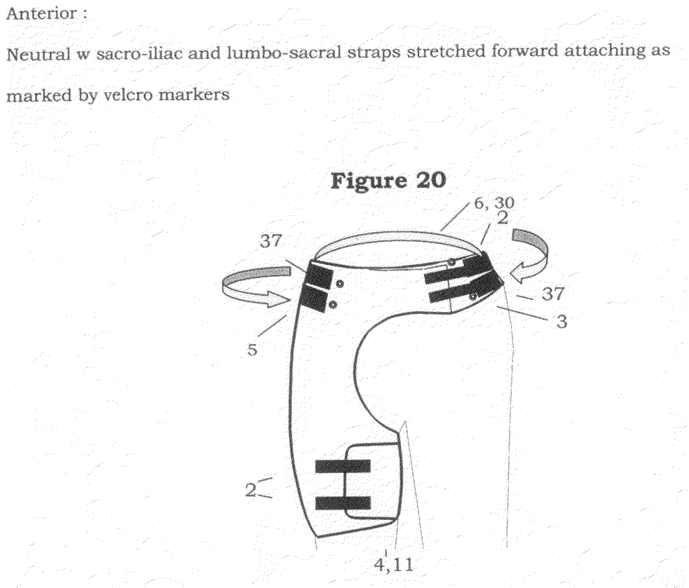

### FIG. 20

### Combined Sacro-Iliac and Lumbo-Sacral Strap Position Reaching Anterior Depiction of the Anterior and Lateral View of the S/I and US Attachments

Each of two neoprene ancillary straps 6 with velcro closures 2,5 or like materials applied one at a time; one anchored on the upper and the other on the lower waist band 3 and drawn around the back to attach on the other side. The exerting pull and of tension is to support the sacro-iliac joints and lumbo-sacral junction. ((FIG. 18) 37,2,4,11

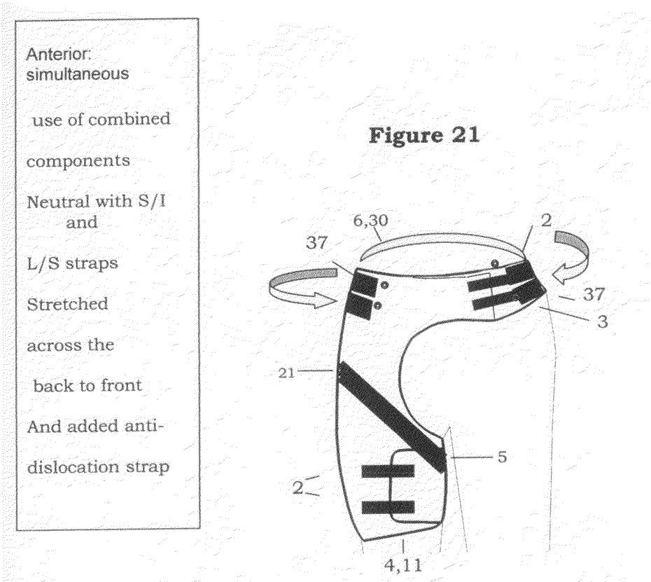

### FIG. 21

### Example of Concomitant Use of Several Components Simultaneously

37,6,30,2,3,5,4,11,21

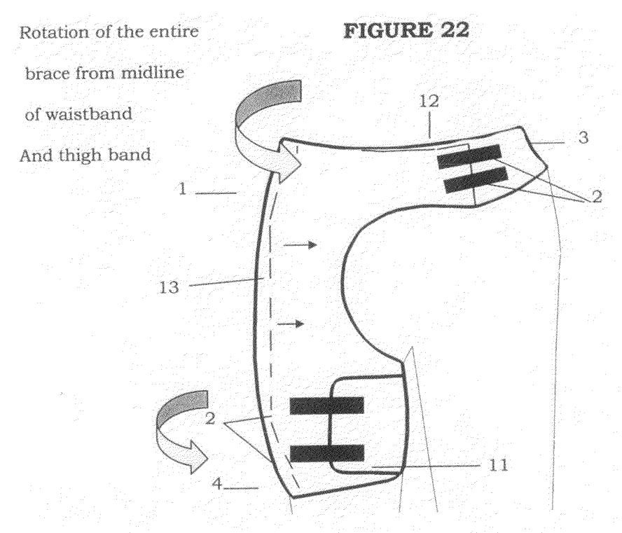

### FIG. 22

### Rotation of the Entire Brace

To fine tune and customize positioning, the entire brace (FIG. 1) can be rotated left or right rotating therefore moving the lateral seam. 1,13,2,4,11,2,3,12

1. materials:
This bracing invention (FIG. 1,2,3,5) with or without ancillary (FIG. 9) neoprene ended straps applied 5 can be worn during any sport, ambulation and ambulatory exercise including walking, power walking, running, jumping, endurance work or normal daily functioning ADL (FIG. 1-22). The brace can be warn by males and females and can be worn above or below clothing. For the individual with bilateral hip problems, two braces for left and right simultaneously or one customized configuration (FIG. 6,4). FIGS. 1-3 depict the embodiment of the hip brace in neutral position, configured for one or 2-sided neoprene body of the brace wraps around the lower torso and thigh. (FIG. 1,5) The body of the brace (FIG. 1,) is constructed of elasticized neoprene or any generation of elasticized fabric with tensile strength, which is secured with velcro closures or like material or closure system. (FIG. 5) The placement and tension of the four velcro straps 2,5,9, and neoprene waist and thigh bands 3,4 as well as the compression onto the entire area covered by the brace dictate the position of the thigh in reference to the pelvis and therefore the hip joint, sacroiliac, and lumbo-sacral spine (FIG. 19-21), 3,2,4,11,12. The posterior portion of the brace stretches from the lumbar spine over the individual's posterior iliac spines, sacrum and reaches distally to contain the sacro-iliac joint (FIG. 3,18) and courses down to the thigh of the affected hip.
A. This bracing invention of claim one, and FIG. 1, using neoprene loop surface on one or both sides (or other elasticized fabric with tensile strength); a sheet of neoprene 1 or like material is configured to have a 1,3 waist band and a thigh band 4,11 (FIG. 1,4). The neoprene or like material can be single sided, whereas the loop surface is on the exterior only with a soft fabric lining or can be double sided loop neoprene UBL surface.
The reversible pattern of the neoprene body (FIG. 1,4) of the brace is shown with the removable alligator (FIG. 8) 10 velcro straps 2, on the ends of the waist and thigh bands 3,4 with both surfaces being receptive to the hook of the velcro 17. The same brace can be flipped for either left or right hip application. This material, single or double is cut to a specific configuration as in the pattern. (FIG. 4) The waist and thigh bands 3,4 are specifically configured to close in the front where adjustment is easiest for the individual, since tension and direction of pull 14 must be made where the straps are most effectively stretched. or like material or other closure system. Thus, once flipped and velcro removed and reversed, it fits the other hip. Or, it can be made singularly as right or left and lined with soft fabric. For close customization for individual people, the neoprene or other elasticized fabric can be cut proportionally as (FIG. 1). The velcro strap length can also be cut.

2. wearing the brace:
This bracing invention of claim 2: Donning: (FIG. 5,4,1) The neoprene body of the brace is wrapped around the lower quadrant of the torso at the waist 3 and thigh 4 of the effected side (FIG. 5). The side view of the right hip showing the position of the midline lateral seam 13 aligned in midline (FIG. 2). This is the beginning position when first donning the brace. 1,2,3,4,13,11,12
A. This bracing invention of claim 2: The narrower band wraps around 12 and overlaps around the waist 3; the wider band wraps and overlaps around the thigh 3,11. Both straps have two velcro closure straps 2 or like closure system (FIG. 1,2,5) Both bands end up attaching on the front of the body, then being secured firmly but comfortably by pressing the velcro hook onto the loop fabric. 1,2,3,4,11,12. The thigh band 4 is then pulled downward and is then re-tightened where the thigh circumference is smaller, (FIG. 5) On release, the exerting and upward force seats the femoral head into the pelvic acetabulum by taking advantage of the elasticized pulling and tensile force of the bracing materials, 14 i.e.; neoprene body of the body of the brace, velcro closure straps yet still allows movement for a normal gait and for exercise all in a supported, advantageous position and alignment.
B. This bracing invention of claim 2:
Ancillary neoprene straps (FIG. 9) have removable alligator velcro on both ends 10. The length of the straps is easily shortened by cutting the neoprene or velcro fabrics to the desired length. These ancillary straps can be used singly or in combination. All velcro 18 is totally removable allowing for precise adjustment and exercise capability (FIG. 7,8,9.) The amount of adjustment and efficacy depends on placement and stretch 14 as well as coverage. They can be a vast variety of widths and lengths. To maintain the position of the added straps 6 or even the waist and thigh bands 3,4 when they overlap, placement of double-sided velcro patches (FIG. 9) applied to the under surface of the strap or band will hold them in place. In order to re-apply the velcro brace to the same strap settings 6, circular patches of velcro 8 can be placed to mark (FIG. 9) where the added strap starts and ends. The hip brace body hereto described is receptive to the adherence of velcro 5,10,17 anywhere on the surface. These ancillary straps serve to fine tune positioning and supportive adjustments by being stretched and attached to various configurations that stabilize, assist, resist, lift, and secure desired position. 38 The compression onto the entire area covered by the brace and other elasticized components provides sensory stimulation to position of the thigh in reference to the pelvis and therefore the hip joint, sacroiliac, and lumbo-sacral spine. (FIG. 1-6,9-22)

3. bracing action:
When the brace is in place 1,2 and the ancillary velcro ended neoprene straps (FIG. 1-3,7,11-21) are in position for the specified chosen purpose, the forces of all of the elasticized components (FIG. 5,10,7,12-22), including the velcro straps that close the brace holding it on the body 2, can coincide to exert the desired pull to result in support, alignment to the hip and sacro-iliac, and lumbo-sacral spine; protection from dislocation, facilitation of exercise resistance or assistance all in a comfortable device. The configuration of the brace and application of additional neoprene straps with velcro closures 17 can be individualized or straight forward anatomical suggestions, not to the exclusion of other possible combinations creating the ideal direction of pull for each individual. (FIG. 11,12-22)
A. This bracing invention of claim 3:
To accomplish moderate positioning into external rotation: (FIG. 10,11) positioning with use of velcro 2 only pulling strategically into external rotation, abduction 14; With the brace body held in neutral (FIG. 1) but the upper thigh velcro hook strap is pulled firmly upward attaching to the neoprene loops at a 45″ angle (FIG. 10), leading the femur into external rotation. For the hip that moderately internally rotates or adducts in flexion, the superior thigh velcro 2,5, is applied at a 45″ angle. The resulting gait will be more toward neutral.
B. This bracing invention of claim 3:
For the dislocating or subluxing hip, especially post surgically; bio-mechanically, the hip must be positioned in external rotation and abduction 1,8,6,12,2,4,11,24,35. starting with the brace in neutral (FIG. 11,2,3,) an ancillary neoprene strap with velcro closures, or like materials is placed on the distal medial surface of the same thigh and drawn up and across the thigh to attach on the posterior waist band. Viewing FIG. 11 from the posterior, the attachment of the ancillary strap with velcro ends is indicated with the dot marker 8. The strap is stretched and pulled upward 21,22 and attached on the posterior waist band on the opposite side. The provocative position for dislocation is prevented by this configuration. 1,3,12,8,6,21,7
C. This bracing invention of claim 3:
For the individual with excessive external rotation; with the goal of bringing the thigh into neutral, starting with brace body held in neutral (FIG. 1,2,3) adding an ancillary neoprene/velcro ended strap 6, to the starting point of posterior/medial thigh, pulling the strap (FIG. 13,14) 23 tension forward anteriorly and upward 14, 20 to attach upon the waist 3 band to the left of center where marked by the dot marker 8,4,11,24,7,6,25 guiding the thigh into adduction and internal rotation Under the strap is a double-sided velcro patch 7 to prevent movement of the strap. (FIG. 13, 14)
D. This bracing invention as in claim 3:
To address specific muscles or muscle groups, to adjust to the strength or lack thereof, the neoprene velcro ended straps or like materials (FIG. 8,9) can be applied to the body of the brace or even to other neoprene ended straps to exert the desired pull and position 14. The combinations of applied positioning and strap locations, direction of pull, 14 and strength of pull are infinite but discernible by the individual, physical therapist, physician, and customized for each individual. This includes using several straps to position in neutral holding that position and performing isotonic and isometric exercise will build strength without imbalance in early rehabilitation. When an ancillary neoprene strap (FIG. 7) 6 or like material with velcro 5 or like material is anchored on the anterior iliac crest by pressing the velcro hook segment onto the neoprene loop and 14 stretched to just above the closure 2 velcro on the anterior thigh of the same side 4 and adhered there by firmly pressing the velcro hook to the loop on the neoprene 5. The direction of pull acts to draw 14 the thigh capitally. This configuration of the body of the brace (FIG. 1,16) acts to assist hip flexion for those with weak hip flexors and/or resistance to hip extension exercise. For abductor assist (FIG. 15); adductor resist a neoprene strap with velcro ends 6 is placed firmly on the lateral iliac crest stretched to the distal end of the lateral side, as marked with the circular marker 8. The tension produced by the pull of the fore mentioned strap assists the leg into abduction for those with weakness of hip abductors. Or, as an exercise, resisting adduction, thereby strengthening the adductors. 27,14.
E. This bracing invention as in claim 3:
To accomplish sacro-iliac joint and lumbo-sacral support bilaterally (18,19,20), two ancillary neoprene straps with velcro ends 6 are stretched 14 across the posterior S/I and L/S joints wrapping anteriorly and adhering 2-4 inches below the waist for the S/I and at the waist arcing above the waist at the posterior; stretched and tightened as tolerated. (FIG. 18,19) onto the medial or medial frontal areas, (FIG. 18,19,20). The hip joint becomes stable, the pelvis levels and sacro-iliac feels secure and is comfortable.
